(19) United States
(12) Patent Application Publication
(10) Pub. No.: US 2025/0017852 A1
(43) Pub. Date: Jan. 16, 2025

(54) HYDROGEL FORMULATION USED AS VITREOUS SUBSTITUTE AND PRODUCTION METHOD THEREOF

(71) Applicant: VSY BIYOTEKNOLOJI VE ILAC SANAYI ANONIM SIRKETI, Istanbul (TR)

(72) Inventors: Efe CAN, Istanbul (TR); Faruk OYTUN, Istanbul (TR); Busra Gizem KAYA, Istanbul (TR)

(73) Assignee: VSY BIYOTEKNOLOJI VE ILAC SANAYI ANONIM SIRKETI, Istanbul (TR)

(21) Appl. No.: 18/712,276

(22) PCT Filed: Nov. 22, 2022
(86) PCT No.: PCT/TR2022/051336

§ 371 Date: May 22, 2024

(30) Foreign Application Priority Data
Nov. 22, 2021 (TR) 2021/018218

(51) Int. Cl. A61K 9/06; A61K 31/727; A61K 31/728; A61K 31/737; A61K 38/39; A61K 47/22

(52) U.S. Cl. CPC A61K 9/06; A61K 31/727; A61K 31/728; A61K 31/737; A61K 38/39; A61K 47/22

(57) ABSTRACT

A hydrogel formulation is developed to be used in ophthalmological applications and as a vitreous substitute. More specifically, a hybrid hydrogel formulation including a cross-linked hyaluronic acid and collagen complex is provided. The hybrid hydrogel formulation can further include a sulfated glycosaminoglycan (sGAG) or ascorbic acid and/or an ascorbic acid derivative.

### CROSS REFERENCE TO THE RELATED APPLICATIONS

This application is the national phase entry of International Application No. PCT/TR2022/051336, filed on Nov. 22, 2022, which is based upon and claims priority to Turkish Patent Application No. 2021/018218, filed on Nov. 22, 2021, the entire contents of which are incorporated herein by reference.

### TECHNICAL FIELD

The present invention relates to a hydrogel formulation developed to be used in ophthalmological applications and as a vitreous substitute, and production method of the said formulation. More specifically, the present invention relates to a hybrid hydrogel formulation comprising cross-linked hyaluronic acid and collagen complex with stable and desirable properties.

### BACKGROUND

The vitreous is a clear gelatinous gel which fills the space between the lens and the retina. It makes up about 80% of the entire volume of the eye. More than 95% by weight of the content of the vitreous body is water; but not all water is in free form. Some of them bind to proteins and glycosaminoglycans. Water is present in a form bound to glycosaminoglycans in a ratio of approximately 15-20%, which ensures the stability of the vitreous structure. Table 1 shows various molecules present in the vitreous [1].

TABLE 1
Biochemical composition of Vitreous

| Groups | Molecule |
|---|---|
| Proteins | Albumin (40%) |
| | Iron-binding protein (30%) (transferrin) |
| | Collagen |
| | Type II (60-70%) |
| | Type IX (25%) |
| | Type V/IX (10-25%) |
| | Type IV (<10%) |
| Glycosaminoglycan | Hyaluronic acid (66-115 microgram/mL |
| (GAG) | concentration) |
| | Chondroitin sulfate |
| | Versican |
| | Type IX collagen |
| | Heparan sulfate |
| Metabolites | Glucose |
| | Lactic acid |
| | Ascorbic acid |
| | Amino acids |
| | Fatty acids unsaturated (50-55%) |
| | Prostaglandins (100 picogram/mL) |
| | PGE2 |
| | PGF2alpha |
| | Prostacyclin |
| | Thromboxane |
| Cells | Hyalocytes |
| | Fibrocytes/fibroblasts |
| | Macrophages |
| | Enzymes and metabolic activity: ACE |

As shown in Table 2. the physical characteristics need to be well known in order to recognize its active role in ocular physiology [1]. The vitreous appears as a structure with its own viscoelastic properties due to a high hyaluronic acid concentration. The collagen-glycosaminoglycan and water frame ensure the transparency of the medium, while acting as support for the vision and accommodation mechanism. The gelatinous structure is adjacent to the posterior hyaloid membrane (vitreous cortex) and it is more dense in the or a serrata. The presence of active molecules provides control over inflammation, proliferation and neovascularization by means of acting as a barrier against infection (bacterial, not viral) [2; 3]. Lastly, the vitreous monitors the processes of drug transport and release within the eye, as well as oxygen and nutrients [4]. These facts show that the vitreous is not only a filling agent, but it also is a physiologically active element in the eye.

TABLE 2
Physical Characteristics of Vitreous.

| | |
|---|---|
| Weight (g) | 4 |
| Density (g/cm³) | 1.0053-1.008 |
| Refractive index | 1.3345-1.3348 |
| Viscosity (cP) | 300-2000 |
| pH | 7.0-7.4 |

Recently, there has been significant progress in vitreoretinal surgery with the development of surgical instruments, drugs, and new surgical techniques. Numerous pathologies such as retinal detachment, diabetic retinopathy, and proliferative vitreoretinopathy require partial or complete removal of the vitreous. This situation has led to the need for vitreous substitutes (retinal tamponades). Ideal vitreous substitutes should have the following characteristics:
It should mimic the natural vitreous
It should be easily manipulated during surgery
It should have viscoelastic characteristics similar to natural vitreous
It should be transparent
It should have a refractive index and density similar to that of natural vitreous.
It should be biologically and chemically inert
It should not cause toxic reactions
It should be biocompatible
It should be easily injectable and aspirable
It should be able to maintain its vitreous substitute task for a minimum of 6 months

In Australian patent document no AU2011215775, an application known in the state of the art, the present invention generally relates to substances that can be used instead of clear, transparent and gelatinous vitreous humor in the vitreous cavity of the eye, behind the lens and in front of the retina, and production and usage methods thereof. In this technique, a composition of materials comprising a hydrogel polymer that can be used in place of vitreous humor is disclosed. This composition comprises a) oxidated hyaluronic acid, b) a dihydrazide which cross-links oxidated hyaluronic acid. The hydrogel polymer exhibits the following properties: a) transparent and colorless; and b) transforming from a liquid state into a gel-matrix at 37 C°. These properties make the material disclosed in the patent useful as a vitreous humor.

In European patent document no EP1144459B2, an application known in the state of the art, the present invention generally relates to cross-linked hyaluronic acid gels obtained by reaction of activated carboxylic groups of hyaluronic acid, of extractive or biosynthetic source, with a polyamine, particularly a linear alkyl diamine. The cross-linked hyaluronic acids can optionally be sulphated or functionalized with hemisuccinyl groups. Such functionalization of hyaluronic acid gels serves many areas. They have many uses such as synovial fluid, vitreous humor, drug release in controlled release systems, vascular prostheses, biohybrid organs, ophthalmic and otological compositions, prostheses, implants and medical devices, thanks to its healing and anti-adhesion properties.

In the international patent document no WO2020163872, an application known in the state of the art, the invention relates to an ophthalmologic composition which can be used as vitreous humor. Here, the vitreous humor composition is a gel (hydrogel or particle) and a therapeutic agent, wherein the therapeutic agent is an antioxidant. The object of the present invention is to create a composition that has the physical properties of the natural vitreous and supports methods that can be used in the treatment of ophthalmological disorders.

In European patent document no EP0185070, an application known in the state of the art, the present invention relates to a method used after vitrectomy in retinal surgery in the field of ophthalmology, which relates to remove partially or completely the vitreous and replace it by injecting cross-linked hyaluronic acid gel.

In United States patent document no US20060159771, an application known in the state of the art, the present invention comprises compositions which are heavier than the vitreous humor and contain one or more glycosaminoglycans (GAGs) and are used in retinal injuries such as retinal detachments (for example, subretinal detachments) and macular holes. These compounds are referred to as “tamponade compositions” for retinal repair and are stated to be useful for repairing retinal damage. New methods are described in the present invention, which include administering a tamponade composition into the vitreous cavity of an eye in a procedure for repairing a retinal insult, for example, an inferior retinal detachment or a macular hole. New tamponade compositions include i) a non-toxic liquid that is denser than aqueous humor, e.g., deuterium-containing heavy water, and ii) one or more GAGs. The new therapeutic methods and compositions disclosed herein have a number of advantages. Tamponade compositions are denser than aqueous humor. Therefore, awkward post-operative positioning of a subject's head is not required to repair inferior retinal tears and macular holes. The new compositions are effective as tamponades due to their high interfacial tension, high surface tension, and high viscosity. Their high surface tension and high interfacial tension provides the new compositions with the ability to press the retinal tissue against the inner wall of the eye, thereby keeping the tissue in place and preventing later detachment.

In European patent document no EP0167363, an application known in the state of the art, the present invention relates to the use of cross-linked GAG or salts thereof cross-linked with a multifunctional epoxy compound, as a therapeutic agent, a medical device or skin cosmetics and medicine, especially for vitreous (glass-like body)

Today, efforts to develop alternative vitreous substitutes that can be used instead of natural vitreous continue. Existing vitreous substitutes currently in use can be classified into three main categories: gases (air, expandable gases), liquids (balanced salt solutions, perfluorocarbon liquids, semi-fluorinated alkanes, silicone oils, etc.), and polymers (natural, semi-synthetic). These vitreous substitutes that are being used have several negative characteristics.

In gas-based substitutes, even though the expanding nature of the gas allows the endotamponade effect to be maintained, in severe cases, a sudden increase in intraocular pressure can cause central retinal artery occlusion. Similarly, patients should avoid higher altitudes to avoid dangerous gas expansion. Perfluorocarbon gas, which has a lower density than vitreous, does not effectively buffer the inferior retina [5]. Although inferior retinal tamponade is clinically achieved using expanding gas, a face-down positioning is required for several days until the inferior detachments are closed and retinal fluid is reabsorbed, which can significantly reduce patient compliance. Adverse effects include gas-induced cataract formation and corneal endothelial changes. In addition, as with air, gases have lower refractive indices compared to the cornea, anterior chamber fluid, and lens. Visual rehabilitation can take up to 1 month.

Currently, perfluorocarbon liquids (PFCLs) are mostly limited to intraoperative use over longer periods of time as a result of their toxicity. Their intravitreal presence in rabbit eyes for more than 2-4 days most probably causes mechanical damage to the cells and it has been shown that it causes irreversible cell damage in the inferior retina 6 days after surgery as a result of almost complete emulsification [6]. In vitro experiments carried out with perfluorodecalin (PFD) and perfluoroperhydrophenanthrene have demonstrated dysregulation in retinal cell growth pattern, neurite loss, and other toxic effects; as a result of this, it has been thought that the high specific gravity of perfluorocarbon liquids compress and disorganize the retinal structure [7; 8].

Cataract formation and emulsification, together with the presence of soft epiretinal membranes and cellular material, are the main problems associated with the use of semi-fluorinated alkanes (SFAs), which are among other liquid substitutes.

Silicon Oil (SO) can pass through retinal detachments more easily than gas, under traction, with a lower surface tension than gas or salt water. The oil is hydrophobic and therefore it does not have the necessary retinal contact. Although SO is clear, its refractive index is 1.4, thereby requiring optical adjustments compared to the vitreous refractive index of 1.3. Considering the low specific gravity of SO, tamponade of the inferior retina is difficult. Emulsification has been shown to be a problem with SO, however, in a few studies, less than 5% of eyes had fat emulsification after surgery [9].

A combination of SO and SFAs does not provide good superior and inferior tamponade at the same time. Bubble formation can be observed with the use of these products, and the bubble, as a whole, acts as a tampon heavier than water. [10].

Major problems with natural and semi-synthetic polymers: Biopolymers are lighter than water, limiting their use as inferior retinal tamponades. In addition, they tend to degrade rapidly in vivo. The biodegradation rate of experimental hyaluronan implants is related to their chemical structure and ranges from 60 to 150 days [11]. Even though hyaluronic acid has been used with limited inflammatory effect, the use of collagen has been limited to inflammation and moderate to severe ocular pain. Collagen gels break as they pass through the syringe during injection and their function as a structural gel is impaired.

### SUMMARY

The objective of the present invention is to provide a hydrogel formulation comprising cross-linked hyaluronic acid and collagen components, developed for use in ophthalmological applications and as a vitreous substitute.

Another objective of the present invention is to develop a hydrogel formulation which is transparent, clear, like natural vitreous, has viscoelastic properties, refractive index and density similar to natural vitreous, is non-toxic, biocompatible, can maintain its function as vitreous substitute for a long time and provides convenience in application.

A further objective of the present invention is to develop a production method suitable for providing the formulation of the present invention.

### DETAILED DESCRIPTION OF THE EMBODIMENTS

The present invention relates to a hydrogel formulation developed to be used in ophthalmological applications and as a vitreous substitute.

The said hydrogel formulation has a hybrid structure; it comprises hyaluronic acid (HA) and collagen, which are contained the most in composition of the vitreous.

In order to obtain a hybrid hydrogel of the present invention, hyaluronic acid and collagen components are cross-linked to each other to form the cross-linked HA-collagen complex and thus the hydrogel. This process takes place by the reaction between the carboxyl groups in the structure of hyaluronic acid and the amine/carboxyl groups in the structure of collagen [18]. During this reaction, first the carboxylic acid groups in the structure of HA are activated with 1-ethyl-3-carbodiimide hydrochloride (EDC·HCl). Then, N-hydroxysuccinimide (NHS), a nucleophilic reagent, converts the activated intermediate product to the NHS ester. These groups that are formed carry out cross-linking by entering into condensation reaction with the amine/carboxyl groups in the structure of collagen. EDC·HCl and NHS reagents used in this process are removed during the purification of the product. Thus, the absence of any synthetic cross-linking agent makes the final product non-toxic.

While hyaluronic acid and collagen can be easily biodegraded in the free state, the hybrid and cross-linked hydrogel form of the invention will ensure that the hyaluronic acid and collagen components remain in the area where they are injected during vitreoretinal surgery for a longer period of time.

Hyaluronic acid in the formulation of the invention is selected from the group comprising a molecular weight of 5000 Da to 5×10⁶ Da and combinations of these molecular weights.

The formulation of the present invention comprises collagen selected from at least one of the group comprising type 1-2-3-4 collagen, hydrolyzed collagen, including animal origin, plant origin, recombinant human collagen or mixtures thereof.

The hydrogel formulation of the invention is a hydrophilic structure with a similar refractive index, pH value, viscosity (cP) and density with natural vitreous.

In the preferred embodiment of the invention, the pH value of the hydrogel formulation is in the range of 7.0 to 7.4, preferably in the range of 7.1 to 7.3.

In the preferred embodiment of the invention, the density of the hydrogel formulation is in the range of 1.000 to 1.008 g/cm³.

In the preferred embodiment of the present invention, the refractive index of the hydrogel formulation is in the range of 1.3345 to 1.334.

In the preferred embodiment of the present invention, the viscosity (cP) of the hydrogel formulation is in the range of 300 to 2000.

Sulfated GAGs (SGAGs), which are included in the formulation of the present invention and are also included in the vitreous structure as versican and IX Collagen, are usually bound to proteins as part of a proteoglycan. It is important in maintaining the structural integrity of the tissue and provides resistance to compression. It also maintains adequate spacing between collagen fibrils and can facilitate the regulation of a wide variety of biological processes, including development, angiogenesis, and blood coagulation, as well as maintaining vitreoretinal adhesion [12]. Sulfated glycosaminoglycans in the formulation of the present invention are at least one of the group comprising chondroitin sulfate (CS), dermatan sulfate (DS), heparan sulfate (HS), heparin (Hep), or mixtures thereof.

The ascorbic acid comprised by the formulation within the scope of the present invention is present in the vitreous body at higher concentrations than the plasma. It has an important role in the age-related liquefaction process and can inhibit neovascularization and increase the proliferation of hyalocytes. It can also prevent rebleeding by inhibiting pathological neovascularization. Recent studies have shown that the antioxidant properties of ascorbic acid can also reduce early cataract formation [13]. Ascorbic acid derivatives in the formulation of the present invention are at least one of the group of L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate and sodium ascorbyl phosphate or mixtures thereof.

The cross-linked gel comprising HA in the present invention is broken down by the hyaluronidase enzymes in the body over time. Inhibiting hyaluronidase enzymes means that the HA-containing gel stays where it is injected for a longer time. It is known that especially the sulfated GAGs and vitamin C derivatives, with the inhibition of hyaluronidase, increase the residence time of the alternative vitreous substitute of the present invention in the body [15].

It is known that sulfated GAGs, which are also used in the said formulation, are good hyaluronidase inhibitors [14]. The inhibition effect is thought to be that negatively charged sulfated GAGs interact electrostatically with the positively charged hyaluronidase enzyme and prevent the enzymes from binding to HA [15]. It is known that vitamin C derivatives in the formulation of the present invention also play an auxiliary role in inhibiting hyaluronidase. In a study, it was reported that Vitamin C inhibits amylase enzymes [16]. In addition, in another study, it was reported that Vitamin C inhibited bovine testicular hyaluronidase [17].

In the present invention, the viscoelastic formulas to be prepared in the buffer solution with disposable sterile syringes are as follows:
1—They comprise linear and/or cross-linked forms of sodium hyaluronate with a molecular weight of 5000 Da-5×10⁶ Da, and is present in the formulation in the concentration range of 0.1 mg/ml-20 mg/ml.
2—They comprise linear and/or cross-linked forms of collagen in the formulation, and it will be present in the formulation in the concentration range of 0.0001 mg/ml-2 mg/ml.
3—Sulfated GAGs in the formulation are selected from a group comprising the following components and their combinations in the concentration range of 0.005 mg/ml-5 mg/ml.
   Chondroitin sulfate
   Dermatan sulfate
   Heparin
   Heparan sulfate
4—Vitamin C derivatives in the formulation are selected from a group comprising the following components and their combinations in the concentration range of 0.002 mg/ml-2 mg/ml.
   L-ascorbic acid
   Tetrahexyldecyl ascorbate
   Ascorbyl glucoside
   Ascorbyl palmitate
   Magnesium ascorbyl palmitate
   Magnesium ascorbyl phosphate
   Calcium ascorbate
   Sodium ascorbate
   Sodium ascorbyl phosphate

A preferred embodiment of the invention is the method for preparing the formulation, which comprises the following steps:
Producing hyaluronic acid-collagen hybrid cross-linked gels
Adding non-crosslinked hyaluronic acid and collagen
Adding sulfated GAG
Adding vitamin C
Mixing for 5-50 hours at a mixing speed of 10-250 rpm
Vacuum degassing of the mixture
Filling the mixture into syringes
Performing vapor sterilization process
Labelling and packaging
Performing ethylene oxide sterilization

In an embodiment of the invention, the concentration value of hyaluronic acid in the formulation is in the range of 1 mg/ml to 20 mg/ml, preferably 1 mg/ml to 5 mg/ml.

In another preferred embodiment of the invention, the formulation of the present invention additionally comprises at least one pharmaceutically and ophthalmologically acceptable excipient.

The above-mentioned non-cross-linked components (e.g. non-cross-linked hyaluronic acid) are used as lubricating agents to facilitate injection.

In the said embodiment of the invention, the invention comprises at least one excipient selected from the group of mineral salts or mixtures thereof.

The following examples are to better illustrate the subject matter of the invention and the subject matter of the invention is not limited to these examples.

### EXAMPLES

Preparation of Cross-Linked Hyaluronic Acid-Collagen hydrogels

2% by weight of sodium hyaluronate (Mw: 3×10⁶ Da), relative to the total solution, was dissolved in deionized water. Then, 0.2% by weight of collagen was added and mixed at 40° C. for 15 minutes. Then, 0.1% by weight of a 100 mM EDC: NHS solution pre-prepared with a mole ratio of 3:1 was added and incubated at 40° C. for 3 hours and left for crosslinking reaction. The resulting gels were then washed 5 times in buffer solution (pH=7.4) to remove unreacted impurities. 3 times by volume was then precipitated in ethyl alcohol. After precipitation, upon removal of alcohol by decanting, HA-Col gels were washed 4 more times with ethyl alcohol and dried under vacuum at 35° C. for 2 days. The formulations were prepared in buffer solution in accordance with the amounts in Table 3 and filled into syringes and sterilized.

TABLE 3
Examples of Formulations

| Components | Formula 1 | Formula 2 | Formula 3 | Formula 4 | Formula 5 |
|---|---|---|---|---|---|
| Cross-linked | 1 | 2 | 3 | 4 | 5 |
| HA-Col | | | | | |
| (mg/ml) | | | | | |
| Non Cross- | 1 | 1 | 1 | 1 | 1 |
| linked HA | | | | | |
| (mg/ml) | | | | | |
| Non Cross- | 0.001 | 0.001 | 0.001 | 0.001 | 0.001 |
| linked | | | | | |
| Collagen | | | | | |
| (mg/ml) | | | | | |
| Chondroitin | 0.25 | 0.25 | 0.25 | 0.25 | 0.25 |
| Sulfate | | | | | |
| (mg/ml) | | | | | |
| Ascorbic | 0.5 | 0.5 | 0.5 | 0.5 | 0.5 |
| Acid | | | | | |
| (mg/ml) | | | | | |

pH, viscosity, density and refractive index values of 5 different formulations with different HA-Collagen cross-linked hydrogel ratios were measured, and the results are summarized in Table 4.

TABLE 4

| Test | Formula 1 | Formula 2 | Formula 3 | Formula 4 | Formula 5 |
|---|---|---|---|---|---|
| pH | 7.12 | 7.14 | 7.21 | 7.16 | 7.22 |
| Viscosity | 420 | 680 | 1050 | 1410 | 1860 |
| (cP) | | | | | |
| Density | 1.0059 | 1.0061 | 1.0060 | 1.0061 | 1.0062 |
| (g/cm³) | | | | | |
| Refractive | 1.3346 | 1.3346 | 1.3346 | 1.3346 | 1.3347 |
| Index | | | | | |

When the results in Table 4 are examined, the pH, viscosity, density and refractive index results of the formulations with different HA-Collagen cross-linked gel ratios are similar to the results of the natural vitreous Therefore, these formulations prepared within the scope of the present invention can be used as alternative vitreous substitutes.

### REFERENCES

[1]. Donati, Simone, et al. “Vitreous substitutes: the present and the future.” BioMed research international 2014 (2014).
[2]. Kleinberg, Teri T., et al. “Vitreous substitutes: a comprehensive review.” Survey of ophthalmology 56.4 (2011): 300-323.
[3]. Los, Leonoor I., et al. “Age-related liquefaction of the human vitreous body: LM and TEM evaluation of the role of proteoglycans and collagen.” Investigative ophthalmology & visual science 44.7 (2003): 2828-2833.
[4]. Filas, Benjamen A., Ying-Bo Shui, and David C. Beebe. “Computational model for oxygen transport and consumption in human vitreous.” Investigative ophthalmology & visual science 54.10 (2013): 6549-6559.
[5]. Pastor, J. Carlos. “Proliferative vitreoretinopathy: an overview.” Survey of ophthalmology 43.1 (1998): 3-18.
[6]. Migliavacca, Luca, Ferdinando Bottoni, and Stefano Miglior. “Experimental short-term tolerance to perfluorodecalin in the rabbit eye: a histopathological study.” Current eye research 17.8 (1998): 828-835.
[7]. Malchiodi-Albedi, Fiorella, et al. “Biocompatibility assessment of silicone oil and perfluorocarbon liquids used in retinal reattachment surgery in rat retinal cultures.” Journal of Biomedical Materials Research: An Official Journal of The Society for Biomaterials, The Japanese Society for Biomaterials, and The Australian Society for Biomaterials and the Korean Society for Biomaterials 60.4 (2002):548-555.
[8]. Matteucci, Andrea, et al. “Biocompatibility assessment of liquid artificial vitreous replacements: relevance of in vitro studies.” Survey of ophthalmology 52.3 (2007): 289-299.
[9]. Azen, Stanley P., et al. “Silicone oil in the repair of complex retinal detachments: a prospective observational multicenter study.” Ophthalmology 105.9 (1998): 1587-1597.
[10]. Heimann, H., T. Stappler, and D. Wong. “Heavy tamponade 1: a review of indications, use, and complications.” Eye 22.10 (2008): 1342-1359.
[11]. Avitabile, Teresio, et al. “Biocompatibility and biodegradation of intravitreal hyaluronan implants in rabbits.” Biomaterials 22.3 (2001): 195-200.
[12]. Buzza, Marguerite S., et al. “Extracellular matrix remodeling by human granzyme B via cleavage of vitronectin, fibronectin, and laminin.” Journal of Biological Chemistry 280.25 (2005): 23549-23558.
[13]. Shui, Ying-Bo, et al. “The gel state of the vitreous and ascorbate-dependent oxygen consumption: relationship to the etiology of nuclear cataracts.” Archives of ophthalmology 127.4 (2009): 475-482.
[14]. Wolf, Robert A., et al. “Heparin inhibits bovine testicular hyaluronidase activity in myocardium of dogs with coronary artery occlusion.” The American journal of cardiology 53.7 (1984): 941-944.
[15]. Girish, K. S., and K. Kemparaju. “Inhibition of Naja naja venom hyaluronidase by plant-derived bioactive components and polysaccharides.” Biochemistry (Moscow)70.8(2005):948-952.
[16]. Abell, Andrew D., Maureen J. Ratcliffe, and Juliet Gerrard. “Ascorbic acid-based inhibitors of α-amylases.” Bioorganic & medicinal chemistry letters 8.13 (1998): 1703-1706.
[17]. Menzel, E. J., and C. Farr. “Hyaluronidase and its substrate hyaluronan: biochemistry, biological activities and therapeutic uses.” Cancer letters 131.1 (1998): 3-11.
[18]. Jia, Weibin, et al. “Fabrication and comprehensive characterization of biomimetic extracellular matrix electrospun scaffold for vascular tissue engineering applications.” Journal of Materials Science 54.15 (2019): 10871-10883.

What is claimed is:

1. A hybrid hydrogel formulation comprising a cross-linked hyaluronic acid and collagen complex for a use as a vitreous substitute.

2. The hybrid_hydrogel formulation according to claim 1, wherein a_molecular weight of hyaluronic acid in the cross-linked hyaluronic acid and collagen complex is in a range of 5000 Da to 5×10⁶ Da.

3. The hybrid hydrogel formulation according to claim 2, wherein the hyaluronic acid is in a concentration range of 0.1 mg/ml to 20 mg/ml.

4. The hybrid hydrogel formulation according to claim 3, wherein collagen in the cross-linked hyaluronic acid and collagen complex is at least one selected from the group consisting of type 1-2-3-4 collagen and hydrolyzed collagen, including animal origin, plant origin, recombinant human collagen, or mixtures thereof.

5. The hybrid hydrogel formulation according to claim 4, wherein the collagen is in a concentration range of 0.0001 mg/ml to 2 mg/ml.

6. The hybrid hydrogel formulation according to claim 1, further comprising a sulfated glycosaminoglycan (sGAG).

7. The hybrid hydrogel formulation according to claim 6, wherein the sGAG is at least one selected from the group consisting of chondroitin sulfate (CS), dermatan sulfate (DS), heparan sulfate (HS), and heparin (Hep) thereof.

8. The hybrid hydrogel formulation according to claim 7, wherein the sGAG is in a concentration range of 0.005 mg/ml to 5 mg/ml.

9. The hybrid hydrogel formulation according to claim 1, further comprising ascorbic acid and/or an ascorbic acid derivative.

10. The hybrid hydrogel formulation according to claim 9, wherein the ascorbic acid derivative is at least one selected from the consisting of L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, and sodium ascorbyl phosphate.

11. The hybrid hydrogel formulation according to claim 10, wherein the ascorbic acid derivative is in a concentration range of 0.002 mg/ml to 2 mg/ml.

12. The hybrid hydrogel formulation according to claim 1, wherein a pH value of the hybrid hydrogel formulation is in a range of 7.0 to 7.4.

13. The hybrid hydrogel formulation according to claim 1, wherein a density of the hybrid hydrogel formulation is in a range of 1.000 g/cm³ to 1.008.

14. The hybrid hydrogel formulation according to claim 1, wherein a refractive index of the hybrid hydrogel formulation is in a range of 1.3345 to 1.3348.

15. The hybrid hydrogel formulation according to claim 1, wherein a viscosity (cP) of the hybrid hydrogel formulation is in a range of 300 to 2000.

16. The hybrid hydrogel formulation according to claim 1, further comprising linear forms of hyaluronic acid and/or collagen.

17. The hybrid hydrogel formulation according to claim 1, comprising the following production method:
producing hyaluronic acid-collagen hybrid cross-linked gels,
adding non-crosslinked hyaluronic acid,
adding sulfated GAG,
adding vitamin C,
mixing for 5-50 hours at a mixing speed of 10-250 rpm,
vacuum degassing of a mixture,
filling the mixture into syringes,
performing a vapor sterilization process.

18. The hybrid hydrogel formulation according to claim 12, wherein the pH value of the hybrid hydrogel formulation is in the range of 7.1 to 7.3.

19. The hybrid hydrogel formulation according to claim 2, wherein a pH value of the hybrid hydrogel formulation is in a range of 7.0 to 7.4.

20. The hybrid hydrogel formulation according to claim 3, wherein a pH value of the hybrid hydrogel formulation is in a range of 7.0 to 7.4.
